(19) United States
(12) Patent Application Publication
(10) Pub. No.: US 2011/0189686 A1
(43) Pub. Date: Aug. 4, 2011

(54) SCREENING FOR MODULATORS OF CYP2B15 AND/OR GPD1 FOR THE TREATMENT OF ACNE, OF SEBORRHOEIC DERMATITIS OR OF HYPERSEBORRHOEA

(75) Inventors: Michel Rivier, Nice (FR); Isabelle Carlavan, Grasse (FR); Jérôme Aubert, Grasse (FR)

(73) Assignee: Galderma Research & Development, Biot (FR)

(21) Appl. No.: 12/991,168

(22) PCT Filed: May 7, 2009
(86) PCT No.: PCT/EP2009/055562

§ 371 Date: Feb. 24, 2011

Related U.S. Application Data
(60) Provisional application No. 61/071,603, filed on May 7, 2008.

(30) Foreign Application Priority Data
Nov. 13, 2008 (FR) 0857716

(51) Int. Cl. C12Q 1/68; C12Q 1/32; C12Q 1/26; C12N 9/04; C12N 9/02; C07H 21/00

(52) U.S. Cl. 435/6.13; 435/26; 435/25; 435/190; 435/189; 536/23.2

(57) ABSTRACT

An in vitro or in vivo method for screening for candidate compounds for the preventive or curative treatment of acne, of seborrhoeic dermatitis or of skin disorders associated with hyperseborrhoea, includes determining the ability of a compound to modulate the expression or the activity of the CYP2B15 and/or glycerol-3-phosphate dehydrogenase 1 (GPD1) proteins.

The invention relates to screening for compounds which modulate the CYP2B15 and/or glycerol-3-phosphate dehydrogenase 1 (GPD1) proteins, that are of use in the treatment of acne, of seborrhoeic dermatitis, and also of skin disorders associated with hyperseborrhoea.

Hyperseborrhoeic greasy skin is characterized by exaggerated secretion and excretion of sebum. Conventionally, a sebum level greater than 200 μg/cm² measured on the forehead is considered to be characteristic of greasy skin. Greasy skin is often associated with a desquamation deficiency, a glistening complexion and a thick skin grain. In addition to these aesthetic disorders, excess sebum can serve as a support for the anarchical development of saprophytic bacterial flora (P. acnes in particular), and cause the appearance of comedones and/or acneic lesions.

This stimulation of sebaceous gland production is induced by androgens.

Acne is, in fact, a chronic disease of the pilosebaceous follicle under hormonal control. Hormone therapy against acne is one treatment possibility for women, the objective being to prevent the effects of androgens on the sebaceous gland. In this context, oestrogens, anti-androgens or agents which reduce the production of androgens by the ovaries or the adrenal gland are generally used. The anti-androgens used for the treatment of acne include, in particular, spironolactone, cyproterone acetate and flutamide. However, these agents have potentially severe side effects. Thus, any pregnancy must be absolutely prevented, in particular because of a risk of feminization for the male foetus. These agents are prohibited in male patients.

Seborrhoeic dermatitis is a common inflammatory skin dermatosis which presents in the form of red plaques covered with greasy, yellowish squames, which are more or less pruriginous, and are predominant in the seborrhoeic areas.

A need therefore exists, for these diseases, to identify mediators downstream of the action of the steroid hormones, and to modulate them, in order to obtain a similar therapeutic profile, but with reduced side effects.

The Applicant has now discovered that the genes encoding the CYP2B15 and glycerol-3-phosphate dehydrogenase 1 (GPD1) proteins are expressed preferentially in rat sebaceous glands in comparison with the epidermis.

The Applicant has more particularly demonstrated that these genes are expressed in a model of animal pharmacology (Fuzzy rat) which is relevant for the acne pathology and hyperseborrhoea (Ye et al, 1997, Skin Pharmacol, 10(5-6):288-97).

More particularly, it demonstrates that the expression of these genes is modulated in vivo at the level of the sebaceous glands following topical treatment with a PPARγ ligand (5-{4-[2-(methylpyridin-2-ylamino)ethoxy]benzyl}thiazolidine-2,4-dione, (S)-2-ethoxy-3-{4-[6-(3-heptyl-1-methylureido)pyridin-2-yl]phenyl}propionic acid or Rosiglitazone, which is 6-(2-methoxyethoxymethoxy)naphthalene-2-carboxylic acid [4′-(2,4-dioxothiazolidin-5-ylmethyl)biphenyl-3-ylmethyl]methylamide, at 1%.

It is moreover, known that treatment with a PPAR agonist induces a large decrease in the size of the sebaceous glands, and a reduction in androgen-induced hyperseborrhoea (WO2007/093747).

Since the genes identified act downstream of the PPAR receptor, they can be used to identify the compounds that are the most active as PPAR modulators, to classify them and to select them. On this basis, it is therefore also proposed to use the CYP2B15 and/or GPD1 genes or the CYP2B15 and/or GPD1 proteins as a marker for screening for candidate PPAR modulators for the treatment of acne, seborrhoeic dermatitis or of a skin disorder associated with hyperseborrhoea. More specifically, the ability of a PPAR modulator to modulate the expression or the activity of CYP2B15 and/or GPD1 or the expression of the gene thereof or the activity of at least one promoter thereof, can be determined.

The term “acne” is intended to mean all the forms of acne, i.e. in particular acne vulgaris, comedonal acne, polymorphous acne, nodulocystic acne, acne conglobata, or else secondary acne such as solar acne, acne medicamentosa or occupational acne. The Applicant also proposes methods of in vitro, in vivo and clinical diagnosis or prognosis based on the detection of the level of expression or of activity of the CYP2B15 and/or GPD1 proteins.

### CYP2B15

The term “CYP2B15” for “cytochrome P450, family 2, subfamily b, polypeptide 15” denotes an enzyme of the cytochrome P450 family, also known as EC 1.14.14.1 or CYPIIB15.

### GPD1

The term “GPD1” denotes glycerol-3-phosphate dehydrogenase 1, also known as EC 1.1.1.8., GPD-C or alternatively GPDH-C.

The GPD1 gene was cloned by Menaya et al., 1995, Biochim. Biophys. Acta 1262:91-94.

Brown et al., 2002 (J. Biol. Chem. 277: 32899-32904) have shown that mice deficient in cytosolic Gpd are phenotypically normal, although they exhibit abnormalities in certain tissues.

In the context of the invention, the term “CYP2B15 gene” or “GPD1 gene” or “CYP2B15 nucleic acid” or “GPD1 nucleic acid” signifies the gene or the nucleic acid sequence which encodes the CYP2B15 and/or glycerol-3-phosphate dehydrogenase 1 proteins. If the target aimed for is preferably the human gene or the expression product thereof, the invention may also make use of cells expressing a heterologous CYP2B15 and/or a heterologous glycerol-3-phosphate dehydrogenase 1, by genomic integration or transient expression of an exogenous nucleic acid encoding the enzyme(s).

In the rat, three alternative transcripts exist for the CYP2B15 genes, encoding three different isoforms of CYP2B15. CYP2B15 cDNA sequences are reproduced in the annexe (SEQ ID No. 1, SEQ ID No. 3 and SEQ ID No. 5). They are, respectively, the sequence XM_{—}001070774 (Genbank), the open reading frame of which contains 1903 base pairs, the sequence XM_{—}001070818 (Genbank), the open reading frame of which contains 1918 base pairs, and the sequence XM_{—}001070869 (Genbank), the open reading frame of which contains 1879 base pairs.

The term “CYP2B15” includes these three isoforms.

A human cDNA sequence of GPD1 is reproduced in the annexe (SEQ ID No. 7). It is the sequence NM_{—}005276 (Genbank), the open reading frame of which contains 2909 base pairs.

### Screening Methods

A subject of the invention is an in vitro or in vivo method for screening for candidate compounds for the preventive and/or curative treatment of acne, of seborrhoeic dermatitis or of any skin disorder associated with hyperseborrhoea, comprising the determination of the ability of a compound to modulate the expression or the activity of the CYP2B15 and/or glycerol-3-phosphate dehydrogenase 1 proteins or the expression of the gene thereof or the activity of at least one of the promoters thereof, said modulation indicating the usefulness of the compound for the preventive or curative treatment of acne, of seborrhoeic dermatitis or of any skin disorder associated with hyperseborrhoea. The method therefore makes it possible to select the compounds capable of modulating the expression or the activity of both and/or either of these enzymes, or the expression of the gene thereof, or the activity of at least one of the promoters thereof.

Preferably, the screening method comprises the determination of the ability of a compound to modulate the expression or the activity of the CYP2B15 protein or the expression of the gene thereof or the activity of at least one of the promoters thereof, and to modulate the expression or the activity of glycerol-3-phosphate dehydrogenase 1 or the expression of the gene thereof or the activity of at least one of the promoters thereof.

More particularly, the subject of the invention is an in vitro method for screening for candidate compounds for the preventive and/or curative treatment of acne, of seborrhoeic dermatitis or of skin disorders associated with hyperseborrhoea, comprising, for both and/or either of the enzymes targeted, the following steps:
a. preparing at least two biological samples or reaction mixtures;
b. bringing one of the samples or reaction mixtures into contact with one or more of the test compounds;
c. measuring the expression or the activity of both and/or either enzyme(s), the expression of the gene thereof or the activity of at least one of the promoters thereof, in the biological samples or reaction mixtures;
d. selecting the compounds for which a modulation of the expression or of the activity of both and/or either enzyme(s), of the expression of the gene thereof or of the activity of at least one of the promoters thereof, is measured in the sample or the mixture treated in b), compared with the untreated sample or with the mixture.

An in vivo screening method can be carried out in any laboratory animal, for example, a rodent. According to one preferred embodiment, the screening method comprises administering the test compound to the animal preferably by topical application, then optionally sacrificing the animal by euthanasia, and taking a sample of an epidermal split, before evaluating the expression of the marker gene(s) in the epidermal split, by any method described herein.

The term “modulation” is intended to mean any effect on the expression or the activity of both and/or either of these enzymes, the expression of the gene or the activity of at least one of the promoters thereof, i.e. optionally a stimulation, but preferably a partial or complete inhibition. Thus, the compounds tested in step d) above preferably inhibit the expression or the activity of the enzymes, the expression of the gene thereof or the activity of at least one of the promoters thereof. The difference in expression obtained with the compound tested, compared with a control carried out in the absence of the compound, is significant starting from 25% or more.

Throughout the present text, unless otherwise specified, the term “expression of a gene” is intended to mean the amount of mRNA expressed;

the term “expression of a protein” is intended to mean the amount of this protein;

the term “activity of a protein” is intended to mean the biological activity thereof;

the term “activity of a promoter” is intended to mean the ability of this promoter to initiate the transcription of the DNA sequence encoded downstream of this promoter (and therefore indirectly the synthesis of the corresponding protein).

The compounds tested may be of any type. They may be of natural origin or may have been produced by chemical synthesis. They may be a library of structurally defined chemical compounds, uncharacterized compounds or substances, or a mixture of compounds.

In particular, the invention is directed towards the use of CYP2B15 and/or GPD1 genes or of the CYP2B15 and/or GPD1 proteins, as a marker for screening for candidate PPAR modulators for the treatment of acne, of seborrhoeic dermatitis or of a skin disorder associated with hyperseborrhoea. More specifically, the ability of a PPAR modulator to modulate the expression or the activity of CYP2B15 and/or GPD1 or the expression of the gene thereof or the activity of at least one of the promoters thereof is determined.

Preferably, the ability of a compound to modulate the expression or the activity of the CYP2B15 protein or the expression of the gene thereof or the activity of at least one of the promoters thereof, and to modulate the expression or the activity of glycerol-3-phosphate dehydrogenase 1 or the expression of the gene thereof or the activity of at least one of the promoters thereof, is determined.

Preferably, the modulator is a PPARγ modulator.

The PPAR modulator is a PPAR agonist or antagonist, preferably a PPAR agonist.

Various techniques can be used to test these compounds and to identify the compounds of therapeutic interest which modulate the expression or the activity of the CYP2B15 and/or glycerol-3-phosphate dehydrogenase 1 proteins.

According to a first embodiment, the biological samples are cells transfected with a reporter gene functionally linked to all or part of the promoter of the gene encoding the CYP2B15 and/or glycerol-3-phosphate dehydrogenase 1 proteins, and step c) described above comprises measuring the expression of said reporter gene.

The reporter gene may in particular encode an enzyme which, in the presence of a given substrate, results in the formation of coloured products, such as CAT (chloramphenicol acetyltransferase), GAL (beta-galactosidase) or GUS (beta-glucuronidase). It may also be the luceriferase gene or the GFP (Green Fluorescent Protein) gene. The assaying of the protein encoded by the reporter gene, or of the activity thereof, is carried out conventionally by colorimetric, fluorometric or chemiluminescence techniques, inter alia.

According to a second embodiment, the biological samples are cells expressing the gene encoding the CYP2B15 and/or glycerol-3-phosphate dehydrogenase 1 proteins, and step c) described above comprises measuring the expression of said gene.

The cell used herein may be of any type. It may be a cell expressing the CYP2B15 and/or GPD1 genes endogenously, for instance a liver cell, an ovarian cell, or better still a sebocyte. Organs of human or animal origin may also be used, for instance the preputial gland, the clitoral gland, or else the sebaceous gland of the skin.

It may also be a cell transformed with a heterologous nucleic acid encoding preferably human, or mammalian, CYP2B15 and/or glycerol-3-phosphate dehydrogenase 1 proteins.

A large variety of host-cell systems may be used, such as, for example, Cos-7, CHO, BHK, 3T3 or HEK293 cells. The nucleic acid may be transfected stably or transiently, by any method known to those skilled in the art, for example by calcium phosphate, DEAE-dextran, liposome, virus, electroporation or microinjection.

In these methods, the expression of the CYP2B15 and/or GPD1 genes or of the reporter gene can be determined by evaluating the level of transcription of said gene, or the level of translation thereof.

The expression “level of transcription of a gene” is intended to mean the amount of corresponding mRNA produced. The expression “level of translation of a gene” is intended to mean the amount of protein produced.

Those skilled in the art are familiar with the techniques for quantitatively or semi-quantitatively detecting the mRNA of a gene of interest. Techniques based on hybridization of the mRNA with specific nucleotide probes are the most common (Northern blotting, RT-PCR (Reverse Transcriptase Polymerase Chain Reaction), quantitative RT-PCR (qRT-PCR), RNase protection). It may be advantageous to use detection labels, such as fluorescent, radioactive or enzymatic agents or other ligands (for example, avidin/biotin).

In particular, the expression of the gene can be measured by real-time PCR or by RNase protection. The term “RNase protection” is intended to mean the detection of a known mRNA among the poly(A)-RNAs of a tissue, which can be carried out using specific hybridization with a labelled probe. The probe is a labelled (radioactive) RNA complementary to the messenger to be sought. It can be constructed from a known mRNA, the cDNA of which, after RT-PCR, has been cloned into a phage. Poly(A)-RNA from the tissue in which the sequence is to be sought is incubated with this probe under slow hybridization conditions in a liquid medium. RNA:RNA hybrids form between the mRNA sought and the antisense probe. The hybridized medium is then incubated with a mixture of ribonucleases specific for single-stranded RNA, such that only the hybrids formed with the probe can withstand this digestion. The digestion product is then deproteinated and repurified, before being analysed by electrophoresis. The labelled hybrid RNAs are detected by autoradiography.

The level of translation of the gene is evaluated, for example, by immunological assaying of the product of said gene. The antibodies used for this purpose may be of polyclonal or monoclonal type. The production thereof involves conventional techniques. An anti-CYP2B15 or GPD1 polyclonal antibody can, inter alia, be obtained by immunization of an animal, such as a rabbit or a mouse, with the whole enzyme. The antiserum is taken and then depleted according to methods known per se to those skilled in the art. A monoclonal antibody can, inter alia, be obtained by the conventional method of Köhler and Milstein (Nature (London), 256: 495-497 (1975)). Other methods for preparing monoclonal antibodies are also known. Monoclonal antibodies can, for example, be produced by expression of a nucleic acid cloned from a hybridoma. Antibodies can also be produced by the phage display technique, by introducing antibody cDNAs into vectors, which are typically filamentous phages which display V-gene libraries at the surface of the phage (for example, fUSE5 for E. coli).

The immunological assaying can be carried out in solid phase or in homogeneous phase; in one step or in two steps; in a sandwich method or in a competition method, by way of nonlimiting examples. According to one preferred embodiment, the capture antibody is immobilized on a solid phase. By way of nonlimiting examples of a solid phase, use may be made of microplates, in particular polystyrene microplates, or solid particles or beads, or paramagnetic beads.

ELISA assays, radioimmunoassays or any other detection technique can be used to reveal the presence of the antigen/antibody complexes formed.

The characterization of the antigen/antibody complexes, and more generally of the isolated or purified, but also recombinant, proteins (obtained in vitro and in vivo) can be carried out by mass spectrometry analysis. This identification is made possible by virtue of the analysis (determination of the mass) of the peptides generated by enzymatic hydrolysis of the proteins (in general, trypsin). In general, the proteins are isolated according to the methods known to those skilled in the art, prior to the enzymatic digestion. The analysis of the peptides (in hydrolysate form) is carried out by separating of the peptides by HPLC (nano-HPLC) based on their physicochemical properties (reverse phase). The determination of the mass of the peptides thus separated is carried out by ionization of the peptides and either by direct coupling with mass spectrometry (electrospray ESI mode), or after deposition and crystallization in the presence of a matrix known to those skilled in the art (analysis in MALDI mode). The proteins are subsequently identified through the use of appropriate software (for example, Mascot).

According to a third embodiment, step a) described above comprises preparing reaction mixtures, each comprising a CYP2B15 and/or GPD1 enzyme and a substrate for the enzyme, and step c) described above comprises measuring the enzymatic activity.

The CYP2B15 and/or GPD1 enzymes can be produced according to customary techniques using Cos-7, CHO, BHK, 3T3 or HEK293 cells. They can also be produced by means of microorganisms such as bacteria (for example, E. coli or B. subtilis), yeasts (for example, Saccharomyces, Pichia) or insect cells, such as Sf9 or Sf21.

The determination of the enzymatic activity of GPD1 preferably comprises the determination of the dehydrogenase activity. A measurement of GPD activity is, for example, described in the article MacDonald and Marshall, 2000, Arch Biochem. Biophys. 384(1):143-53.

The compounds selected by means of the screening methods defined herein can subsequently be tested on other in vitro models and/or in vivo models (in animals or humans) for their effects on acne, seborrhoeic dermatitis or skin disorders associated with hyperseborrhoea.

The following examples illustrate the invention without limiting the scope thereof.

### EXAMPLES

### A. Experimental Data Concerning the CYP2B15 Enzyme

### Example 1

### Expression of the CYP2B15 Protein in Rat Epidermis

### Fuzzy Rat Epidermal Split Expression Data

The studies are carried out in female Fuzzy rats (Hsd: FUZZY-fz) 10 weeks old at the beginning of the study. The animals are treated at a dose of 1% (PPARg agonist Rosiglitazone in solution in acetone) once a day for 8 days. Two hours after the final treatment, the animals are sacrificed by euthanasia and the skin on the back is removed. After incubation in dispase, the epidermis carrying the sebaceous glands is detached from the dermis (epidermal split). After grinding of the samples, the mRNA is prepared using Qiagen columns, in accordance with the suppliers' instructions. The material thus prepared is subjected to large-scale transcriptome analysis on an Affymetrix platform. The data are subsequently standardized and, after statistical analysis, the results produced are expressed in arbitrary expression units (see below) accompanied, for each piece of data, by a statistical value for presence of the transcript (presence=1; absence=0).

TABLE 1
Measurement of the expression of CYP2B15 in an
epidermal split after 8 days of topical treatment of
FUZZY rat females with a PPARγ agonist (Rosiglitazone) at 1%

| | | | | Significance | Significance |
|---|---|---|---|---|---|
| | | Expression | | of the | of the |
| | | under | Expression | expression* | expression* |
| | | the | after | under | after |
| | | control | treatment | the | treatment |
| Affymetrix | | condition | with 1% | control | with 1% |
| identifier | Gene name | (DMSO) | Rosiglitazone | condition | Rosiglitazone |
| 1387722_at | cytochrome | 120 | 35 | 1 | 1 |
| | P450, | | | | |
| | family 2, | | | | |
| | subfamily | | | | |
| | b, | | | | |
| | polypeptide | | | | |
| | 15 | | | | |

*indicator of the significance of the expression of the gene analysed in the sample indicated: presence (=1) or absence (=0).

### Example 2

### Data for Expression in the Rat Sebaceous Gland after Treatment with a PPARgamma Receptor Agonist

### Materials and Methods:

### Animals: Species: rat

Strain: Ico:HSd:FUZZY-fz
Gender: female
Age: 10 weeks
   Number per batch: 40 (8 animals per group)
   Treatment: Route of administration: topical
Compound/batch: PPARgamma agonists:
A: 5-{4-[2-(methylpyridin-2-ylamino)ethoxy]benzyl}thiazolidine-2,4-dione
B: 2-(methoxyethoxymethoxy)naphthalene-2-carboxylic acid [4′-(2,4-dioxothiazolidin-5-ylmethyl)biphenyl-3-ylmethyl]methylamide or rosiglitazone
C: (S)-2-ethoxy-3-{4-[6-(3-heptyl-1-methylureido)pyridin-2-yl]phenyl}propionic acid
Doses: 1%
Carrier: acetone (001)
Duration: 96 hours
   Method of evaluation: The animals are weighed at the beginning and at the end of the study. Skin biopsies are taken (6 samples of skin excised per rat) in order to analyze the expression of the genes (RNA extraction, reverse transcriptase and real-time PCR). The samples are stored at 4° C. overnight before incubation in 1 M sodium bromide (NaBr) for 2 hours at 37° C. After incubation, the samples are separated into epidermis or dermis. The epidermal samples are stored at 20° C. Under these conditions, the sebaceous glands are in the epidermal split. PCRs are carried out, beginning with the cDNAs originating from the epidermal splits containing sebaceous glands from control rats or rats treated with a PPARγ agonist: the mRNA is extracted using a column and quantified. The quality of the mRNAs is measured and is represented by the 18S/28S ratio. The results are standardized with respect to 18S, expressed as relative induction versus untreated animals (carrier group). The statistical analysis is obtained using internal software based on a modified Monte Carlo statistical analysis.

### Results:

| CYP2B15 | Relative induction- kinetics (hours) | | | | |
|---|---|---|---|---|---|
| Treatment | 0 | 8 | 24 | 48 | 96 |
| A | 1 | 1.56 | 0.68 | 0.29 | 0.08 |
| B | 1 | 1.05 | 1.25 | 0.63 | 0.83 |
| C | 1 | 0.61 | 0.18 | 0.11 | 0.04 |

### B. Experimental Data Concerning the GPD1 Enzyme

### Example 3

### Expression of the GPD1 Protein in Rat Epidermis

### Fuzzy Rat Epidermal Split Expression Data

The studies are carried out in female Fuzzy rats (Hsd: FUZZY-fz) 10 weeks old at the beginning of the study. The animals are treated at a dose of 1% (PPARg agonist Rosiglitazone in solution in acetone) once a day for 8 days. Two hours after the final treatment, the animals are sacrificed by euthanasia and the skin on the back is removed. After incubation in dispase, the epidermis carrying the sebaceous glands is detached from the dermis (epidermal split). After grinding of the samples, the mRNA is prepared using Qiagen columns, in accordance with the suppliers' instructions. The material thus prepared is subjected to large-scale transcriptome analysis on an Affymetrix platform. The data are subsequently standardized and, after statistical analysis, the results produced are expressed in arbitrary expression units (see below) accompanied, for each piece of data, by a statistical value for presence of the transcript (presence=1; absence=0).

TABLE 3
Measurement of the expression of GPD1 in an
epidermal split after 8 days of topical treatment of
FUZZY rat females with a PPARγ agonist (Rosiglitazone) at 1%

| | | | | | Significance |
|---|---|---|---|---|---|
| | | Expression | | Significance | of the |
| | | under | Expression | of the | expression* |
| | | the | after | expression* | after |
| | | control | treatment | under the | treatment |
| Affymetrix | Gene | condition | with 1% | control | with 1% |
| identifier | name | (DMSO) | Rosiglitazone | condition | Rosiglitazone |
| 1371363_at | glycerol- | 66 | 243 | 1 | 1 |
| | 3- | | | | |
| | phosphate | | | | |
| | dehydrogenase 1 | | | | |

*indicator of the significance of the expression of the gene analysed in the sample indicated: presence (=1) or absence (=0).

### Example 4

### Data for Expression in The Rat Sebaceous Gland after Treatment with a PPARgamma Receptor Agonist

### Materials and Methods:

### Animals: Species: rat

Strain: Ico:Hsd FUZZY-fz
Gender: female
Age: 10 weeks
   Number per batch: 40 (8 animals per group)
   Treatment: Route of administration: topical
Compound/batch: PPARgamma agonists:
A: 5-{4-[2-(methylpyridin-2-ylamino)ethoxy]benzyl}thiazolidine-2,4-dione
B: 2-(methoxyethoxymethoxy)naphthalene-2-carboxylic acid [4′-(2,4-dioxothiazolidin-5-ylmethyl)biphenyl-3-ylmethyl]methylamide or rosiglitazone
C: (S)-2-ethoxy-3-{4-[6-(3-heptyl-1-methylureido)pyridin-2-yl]phenyl}propionic acid
Doses: 1%
Carrier: acetone (001)
Duration: 96 hours
   Method of evaluation: The animals are weighed at the beginning and at the end of the study. Skin biopsies are taken (6 samples of skin excised per rat) in order to analyze the expression of the genes (RNA extraction, reverse transcriptase and real-time PCR). The samples are stored at 4° C. overnight before incubation in 1 M sodium bromide (NaBr) for 2 hours at 37° C. After incubation, the samples are separated into epidermis or dermis. The epidermal samples are stored at 20° C. Under these conditions, the sebaceous glands are in the epidermal split. PCRs are carried out, beginning with the cDNAs originating from the epidermal splits containing sebaceous glands from control rats or rats treated with a PPARγ agonist: the mRNA is extracted using a column and quantified. The quality of the mRNAs is measured and is represented by the 18S/28S ratio. The results are standardized with respect to 18S, expressed as relative induction versus untreated animals (carrier group). The statistical analysis is obtained using internal software based on a modified Monte Carlo statistical analysis.

### Results:

| GPD1 | Relative induction- kinetics (hours) | | | | |
|---|---|---|---|---|---|
| Treatment | 0 | 8 | 24 | 48 | 96 |
| A | 1 | 18.52 | 12.53 | 6.93 | 3.42 |
| B | 1 | 6.56 | 3.66 | 3.02 | 0.91 |
| C | 1 | 7.59 | 11.56 | 7.50 | 3.44 |

1.-16. (canceled)

17. An in vitro or in vivo method for screening for candidate compounds for the preventive and/or curative treatment of acne, of seborrhoeic dermatitis or of skin disorders associated with hyperseborrhoea, comprising determining the ability of a compound to modulate the expression or the activity of the CYP2B15 and/or glycerol-3-phosphate dehydrogenase 1 (GPD1) proteins or the expression of the gene thereof, or the activity of at least one of the promoters thereof.

18. An in vitro method for screening for candidate compounds for the preventive and/or curative treatment of acne, of seborrhoeic dermatitis or of skin disorders associated with hyperseborrhoea as defined by claim 17, comprising the following steps:
a. preparing at least two biological samples or reaction mixtures;
b. contacting one of the samples or reaction mixtures with one or more of the test compounds;
c. measuring the expression or the activity of both and/or either enzyme(s), the expression of the gene thereof or the activity of at least one of the promoters thereof, in the biological samples or reaction mixtures; and
d. selecting the compounds for which a modulation of the expression or of the activity of both and/or either enzyme(s), or a modulation of the expression of the gene thereof or a modulation of the activity of at least one of the promoters thereof, is measured in the sample or the mixture treated in b), compared with the untreated sample or with the untreated mixture.

19. The in vitro method as defined by claim 18, wherein the compounds selected in step d) inhibit the expression or the activity of both and/or either enzyme(s), the expression of the gene thereof or the activity of at least one of the promoters thereof.

20. The in vitro method as defined by claim 18, wherein the biological samples are cells transfected with a reporter gene functionally linked to all or part of the promoter of the gene encoding the CYP2B15 and/or glycerol-3-phosphate dehydrogenase 1 proteins, and step c) comprises measuring the expression of said reporter gene.

21. The in vitro method as defined by claim 18, wherein the biological samples comprise cells expressing the gene(s) encoding the CYP2B15 and/or glycerol-3-phosphate dehydrogenase 1 protein(s), and step c) comprises measuring the expression of said gene.

22. The in vitro method as defined by claim 20, wherein the cells comprise sebocytes.

23. The in vitro method as defined by claim 21, wherein the cells comprise cells transformed with a heterologous nucleic acid encoding the CYP2B15 and/or glycerol-3-phosphate dehydrogenase 1 proteins.

24. The in vitro method as defined by claim 18, wherein the expression of the gene is determined by measuring the level of transcription of said gene.

25. The in vitro method as defined by claim 18, wherein the expression of the gene is determined by measuring the level of translation of said gene.

26. The in vitro method as defined by claim 18, wherein step a) comprises preparing reaction mixtures, each comprising a CYP2B15 and/or glycerol-3-phosphate dehydrogenase 1 enzyme and a substrate for the enzyme, and step c) comprises measuring the enzymatic activity.

27. The in vitro or in vivo method as defined by claim 17, comprising determining the ability of a compound to modulate the expression or the activity of the CYP2B15 protein or the expression of the gene thereof or the activity of at least one of the promoters thereof, and to modulate the expression or the activity of glycerol-3-phosphate dehydrogenase 1 or the expression of the gene thereof or the activity of at least one of the promoters thereof.

28. A marker for screening for candidate PPAR modulators for the treatment of acne, of seborrhoeic dermatitis or of a skin disorder associated with hyperseborrhoea, comprising the CYP2B15 and/or glycerol-3-phosphate dehydrogenase 1 genes or proteins.

29. The marker as defined by claim 28, for determining the ability of a PPAR modulator to modulate the expression or the activity of the CYP2B15 and/or glycerol-3-phosphate dehydrogenase 1 proteins or the expression of the gene thereof or the activity of at least one of the promoters thereof.

30. The marker as defined by claim 29, for determining the ability of a compound to modulate the expression or the activity of the CYP2B15 protein or the expression of the gene thereof or the activity of at least one of the promoters thereof, and to modulate the expression or the activity of glycerol-3-phosphate dehydrogenase 1 or the expression of the gene thereof or the activity of at least one of the promoters thereof.

31. The marker as defined by claim 28, wherein the PPAR modulator comprises a PPARγ modulator.

32. The marker as defined by claim 28, wherein the modulator comprises a PPAR receptor agonist.


SEQUENCE LISTING (8 sequences)

SEQ ID NO: 1 - DNA, 1903 bp, Rattus norvegicus
gtggttacaccaggaccatggagcccagtatcttgctcctccttgctctcctcgtgggcttcttgttactcttagtcaggggacacccaaagtcccgtggcaacttcccaccaggacctcgtccccttcccctcttggggaacctcctgcagttggacagagggggcctcctcaattccttcatgcagcttcgagaaaaatatggagatgtgttcacagtacacctgggaccaaggcctgtggtcatgctatgtgggacagacaccataaaggaggctctggtgggccaagctgaggatttctctggtcggggaacaatcgctgtgattgagccaatcttcaaggaatatggtgtgatctttgccaatggggaacgctggaaggcccttcggcgattctctctggctaccatgagagactttgggatgggaaagaggagtgtggaagaacggattcaggaggaagcccaatgtttggtggaggaactgcggaaatcccagggagccccactggatcccaccttcctcttccagtgcatcacagccaacatcatctgctccattgtgtttggagagcgctttgactacacagaccgccagttcctgcgcctgttggagctgttctaccggaccttttccctcctaagttcattctccagccaggtgtttgagttcttctctgggttcctgaaatactttcctggtgcccacagacaaatctccaaaaacctccaggaaatcctcgattacattggccatattgtggagaagcacagggccaccttagacccaagcgctccacgagacttcatcgacacttaccttctgcgcatggagaaggtgagtcctgcatggatgagagaggagaagtcgaaccaccacacagtgttccatcatgagaacctcatgatctccctgctctctctcttctttgctggcactgagaccagcagcaccacactccgctatggtttcctgctgatgctcaagtacccccatgtcgcagagaaagtccaaaaggagattgatcaggtgatcggctcacaccggctaccaacccttgatgaccgcagtaaaatgccatacactgatgcagttatccatgagattcagaggttttcagatcttgtccctattggagtaccacacagagtcaccaaagacaccatgttccgagggtacctgcttcccaagaacactgaagtgtaccccatcctgagttcagctctccatgacccacagtactttgaccacccagacagcttcaatcctgaacacttcctggatgccaatggggcactgaaaaagagtgaagctttcatgcccttctccacaggaaagcgcatttgtcttggcgaaggcattgcccgaaatgaattgttcctcttcttcaccaccatcctccagaacttctctgtgtcaagccatttggctcccaaggacattgacctcacgcccaaggagagtggcattggaaaaatacctccaacgtaccagatctgcttctcagctcggtgatccggctgaggcagccatgtgccccagttctgttgggaatggcctcatgtttctgcctctgggggacctgctgaaaaccaggctcaaggccactgctcacatcttcctattgcagttctccaaagtcccaaggctttttcttattcctgtgaatggcactgaagaagtcaatcggctgtcttattttgacatgtgaacagagatttcatgagtccacatctcatgctgagtcacttccctcttcctcctaacagcccatgtccccagttatcagccctccatggtctgtgatctgtgctaatggactctgtatatggtctcagtgctatgtctacagacttacatagtatgtatggttcaggtaaacagaatcacagagtgtgtga

SEQ ID NO: 2 - protein, 499 aa, Rattus norvegicus
MEPSILLLLALLVGFLLLLVRGHPKSRGNFPPGPRPLPLLGNLLQLDRGGLLNSFMQLREKYGDVFTVHLGPRPVVMLCGTDTIKEALVGQAEDFSGRGTIAVIEPIFKEYGVIFANGERWKALRRFSLATMRDFGMGKRSVEERIQEEAQCLVEELRKSQGAPLDPTFLFQCITANIICSIVFGERFDYTDRQFLRLLELFYRTFSLLSSFSSQVFEFFSGFLKYFPGAHRQISKNLQEILDYIGHIVEKHRATLDPSAPRDFIDTYLLRMEKVSPAWMREEKSNHHTVFHHENLMISLLSLFFAGTETSSTTLRYGFLLMLKYPHVAEKVQKEIDQVIGSHRLPTLDDRSKMPYTDAVIHEIQRFSDLVPIGVPHRVTKDTMFRGYLLPKNTEVYPILSSALHDPQYFDHPDSFNPEHFLDANGALKKSEAFMPFSTGKRICLGEGIARNELFLFFTTILQNFSVSSHLAPKDIDLTPKESGIGKIPPTYQICFSAR

SEQ ID NO: 3 - DNA, 1918 bp, Rattus norvegicus
gtggttacaccaggaccatggagcccagtatcttgctcctccttgctctcctcgtgggcttcttgttactcttagtcaggggacacccaaagtcccgtggcaacttcccaccaggacctcgtccccttcccctcttggggaacctcctgcagttggacagagggggcctcctcaattccttcatgcagcttcgagaaaaatatggagatgtgttcacagtacacctgggaccaaggcctgtggtcatgctatgtgggacagacaccataaaggaggctctggtgggccaagctgaggatttctctggtcggggaacaatcgctgtgattgagccaatcttcaaggaatatggtgtgatctttgccaatggggaacgctggaaggcccttcggcgattctctctggctaccatgagagactttgggatgggaaagaggagtgtggaagaacggattcaggaggaagcccaatgtttggtggaggaactgcggaaatcccagggagccccactggatcccaccttcctcttccagtgcatcacagccaacatcatctgctccattgtgtttggagagcgctttgactacacagaccgccagttcctgcgcctgttggagctgttctaccggaccttttccctcctaagttcattctccagccaggtgtttgagttcttctctgggttcctgaaatactttcctggtgcccacagacaaatctccaaaaacctccaggaaatcctcgattacattggccatattgtggagaagcacagggccaccttagacccaagcgctccacgagacttcatcgacacttaccttctgcgcatggagaaggagaagtcgaaccaccacacagtgttccatcatgagaacctcatgatctccctgctctctctcttctttgctggcactgagaccagcagcaccacactccgctatggtttcctgctgatgctcaagtacccccatgtcgcagagaaagtccaaaaggagattgatcaggtgatcggctcacaccggctaccaacccttgatgaccgcagtaaaatgccatacactgatgcagttatccatgagattcagaggttttcagatcttgtccctattggagtaccacacagagtcaccaaagacaccatgttccgagggtacctgcttcccaagaacactgaagtgtaccccatcctgagttcagctctccatgacccacagtactttgaccacccagacagcttcaatcctgaacacttcctggatgccaatggggcactgaaaaagagtgaagctttcatgcccttctccacaggtgaggcagaattgcttataatttatcagaactccattggaaagcgcatttgtcttggcgaaggcattgcccgaaatgaattgttcctcttcttcaccaccatcctccagaacttctctgtgtcaagccatttggctcccaaggacattgacctcacgcccaaggagagtggcattggaaaaatacctccaacgtaccagatctgcttctcagctcggtgatccggctgaggcagccatgtgccccagttctgttgggaatggcctcatgtttctgcctctgggggacctgctgaaaaccaggctcaaggccactgctcacatcttcctattgcagttctccaaagtcccaaggctttttcttattcctgtgaatggcactgaagaagtcaatcggctgtcttattttgacatgtgaacagagatttcatgagtccacatctcatgctgagtcacttccctcttcctcctaacagcccatgtccccagttatcagccctccatggtctgtgatctgtgctaatggactctgtatatggtctcagtgctatgtctacagacttacatagtatgtatggttcaggtaaacagaatcacagagtgtgtga

SEQ ID NO: 4 - protein, 504 aa, Rattus norvegicus
MEPSILLLLALLVGFLLLLVRGHPKSRGNFPPGPRPLPLLGNLLQLDRGGLLNSFMQLREKYGDVFTVHLGPRPVVMLCGTDTIKEALVGQAEDFSGRGTIAVIEPIFKEYGVIFANGERWKALRRFSLATMRDFGMGKRSVEERIQEEAQCLVEELRKSQGAPLDPTFLFQCITANIICSIVFGERFDYTDRQFLRLLELFYRTFSLLSSFSSQVFEFFSGFLKYFPGAHRQISKNLQEILDYIGHIVEKHRATLDPSAPRDFIDTYLLRMEKEKSNHHTVFHHENLMISLLSLFFAGTETSSTTLRYGFLLMLKYPHVAEKVQKEIDQVIGSHRLPTLDDRSKMPYTDAVIHEIQRFSDLVPIGVPHRVTKDTMFRGYLLPKNTEVYPILSSALHDPQYFDHPDSFNPEHFLDANGALKKSEAFMPFSTGEAELLIIYQNSIGKRICLGEGIARNELFLFFTTILQNFSVSSHLAPKDIDLTPKESGIGKIPPTYQICFSAR

SEQ ID NO: 5 - DNA, 1879 bp, Rattus norvegicus
gtggttacaccaggaccatggagcccagtatcttgctcctccttgctctcctcgtgggcttcttgttactcttagtcaggggacacccaaagtcccgtggcaacttcccaccaggacctcgtccccttcccctcttggggaacctcctgcagttggacagagggggcctcctcaattccttcatgcagcttcgagaaaaatatggagatgtgttcacagtacacctgggaccaaggcctgtggtcatgctatgtgggacagacaccataaaggaggctctggtgggccaagctgaggatttctctggtcggggaacaatcgctgtgattgagccaatcttcaaggaatatggtgtgatctttgccaatggggaacgctggaaggcccttcggcgattctctctggctaccatgagagactttgggatgggaaagaggagtgtggaagaacggattcaggaggaagcccaatgtttggtggaggaactgcggaaatcccagggagccccactggatcccaccttcctcttccagtgcatcacagccaacatcatctgctccattgtgtttggagagcgctttgactacacagaccgccagttcctgcgcctgttggagctgttctaccggaccttttccctcctaagttcattctccagccaggtgtttgagttcttctctgggttcctgaaatactttcctggtgcccacagacaaatctccaaaaacctccaggaaatcctcgattacattggccatattgtggagaagcacagggccaccttagacccaagcgctccacgagacttcatcgacacttaccttctgcgcatggagaaggagaagtcgaaccaccacacagtgttccatcatgagaacctcatgatctccctgctctctctcttctttgctggcactgagaccagcagcaccacactccgctatggtttcctgctgatgctcaagtacccccatgtcgcagagaaagtccaaaaggagattgatcaggtgatcggctcacaccggctaccaacccttgatgaccgcagtaaaatgccatacactgatgcagttatccatgagattcagaggttttcagatcttgtccctattggagtaccacacagagtcaccaaagacaccatgttccgagggtacctgcttcccaagaacactgaagtgtaccccatcctgagttcagctctccatgacccacagtactttgaccacccagacagcttcaatcctgaacacttcctggatgccaatggggcactgaaaaagagtgaagctttcatgcccttctccacaggaaagcgcatttgtcttggcgaaggcattgcccgaaatgaattgttcctcttcttcaccaccatcctccagaacttctctgtgtcaagccatttggctcccaaggacattgacctcacgcccaaggagagtggcattggaaaaatacctccaacgtaccagatctgcttctcagctcggtgatccggctgaggcagccatgtgccccagttctgttgggaatggcctcatgtttctgcctctgggggacctgctgaaaaccaggctcaaggccactgctcacatcttcctattgcagttctccaaagtcccaaggctttttcttattcctgtgaatggcactgaagaagtcaatcggctgtcttattttgacatgtgaacagagatttcatgagtccacatctcatgctgagtcacttccctcttcctcctaacagcccatgtccccagttatcagccctccatggtctgtgatctgtgctaatggactctgtatatggtctcagtgctatgtctacagacttacatagtatgtatggttcaggtaaacagaatcacagagtgtgtga

SEQ ID NO: 6 - protein, 491 aa, Rattus norvegicus
MEPSILLLLALLVGFLLLLVRGHPKSRGNFPPGPRPLPLLGNLLQLDRGGLLNSFMQLREKYGDVFTVHLGPRPVVMLCGTDTIKEALVGQAEDFSGRGTIAVIEPIFKEYGVIFANGERWKALRRFSLATMRDFGMGKRSVEERIQEEAQCLVEELRKSQGAPLDPTFLFQCITANIICSIVFGERFDYTDRQFLRLLELFYRTFSLLSSFSSQVFEFFSGFLKYFPGAHRQISKNLQEILDYIGHIVEKHRATLDPSAPRDFIDTYLLRMEKEKSNHHTVFHHENLMISLLSLFFAGTETSSTTLRYGFLLMLKYPHVAEKVQKEIDQVIGSHRLPTLDDRSKMPYTDAVIHEIQRFSDLVPIGVPHRVTKDTMFRGYLLPKNTEVYPILSSALHDPQYFDHPDSFNPEHFLDANGALKKSEAFMPFSTGKRICLGEGIARNELFLFFTTILQNFSVSSHLAPKDIDLTPKESGIGKIPPTYQICFSAR

SEQ ID NO: 7 - DNA, 2909 bp, Homo sapiens
gcactgagccggctcaggcagagacgcggcaccatggctagcaagaaagtctgcattgtaggctccgggaactggggctcagccatcgccaagatcgtgggtggcaatgcagcccagctggcacagtttgacccacgggtgaccatgtgggtatttgaggaagacattggaggcaaaaagctgactgagatcatcaacacgcagcatgagaatgtcaaatacctgccagggcacaagttgcccccaaatgtggtggctgtcccagatgtggtccaggctgcagaggatgctgacatcctgatctttgtggtgccccatcagttcatcggcaagatctgtgaccagctcaagggccatctgaaggcaaacgccactggcatatctcttattaagggggtagacgagggccccaatgggctgaagctcatctcggaagtgattggggagcgcctcggcatccccatgagtgtgctgatgggggccaacattgccagcgaggtggctgatgagaagttctgtgagacaaccattggctgcaaggacccggcccagggacaactcctgaaagagctgatgcagacaccaaacttccgtatcacagtggtgcaagaggtggacacagtagagatctgtggagccttaaagaatgtagtggccgtgggggctggcttctgtgatggcctgggctttggcgacaacaccaaggcggcagtgatccggctgggactcatggagatgatagccttcgccaagctcttctgcagtggccctgtgtcctctgccaccttcttggagagctgtggtgttgctgacctgatcactacctgctatggagggcggaaccggaaagtggctgaggcctttgcgcgtacaggaaagtccattgagcagctggagaaagagttgctgaatgggcagaaactgcaggggcccgagacagcccgggagctatacagcatcctccagcacaagggcctggtagacaagtttcccttgttcatggctgtgtacaaggtgtgctacgagggccagccagtgggtgaattcatccactgcctgcagaatcatccagaacatatgtgagtggggccagggcccaggccaggccgcttttttaccccagcggagaccagcagaagcctggggtacctagtcaccaggatctccaggactcccagggagcagagtcttctcatcttttcactggaggacaggaggctatggggcccagctacgcacctggagatcctgaactgtcaagccactggcagcctcatgccaccacatttgccagaaatgcagttgccctgtccctctccagatgtggggctttctccatatcctctgggaggggtggaatcaagccccagtgctgcctgcttggtggcgggggtgatgtatgtggagaagggttgggggagaggccggtagggcaggggctgctagtggctgtctcacatacaccagtaatcctgttaaagggctgaagaagtatcttagccacaggagcgatgaggcaaggattgtcagggaggggtctgggcttctgagctgatgcaggccccaaggacccctttgctgacctctgccaggacccacacagcttcgatggatctcagtgtttgttaacaaaatacaaagatctcaaacaaaccccttttagcttctcctagcaacatctgtgtcctcagaaacctctggttctcccccttccccctcccccaggctgccctggcacccaaattgctgccatgctggcatctgtagctcggtggcttgacattctccccagggacttcccggttcctagttctttgccagctcctccccactctgtgcgacctttccaagccttccctcaccctccccgccagcaccctctttggggagcaggaacttaatctgctgacagagctacacctttcataacaggctcagatcactcagctccctgtgacctttgtattcacccaggcttccttctcagtagctctagctgggggaggtgtcagctgggcccctcctgtgctatctccccagaggacatccctgactccacccctttcttcctcccaaactctgcacctcttcctcagctgctccagaccggccagggtaaccagctaaccatgcctggcatctggaagccagcaggccagagggtggcccaaaggctgatgaacggtagggaagggtgagcagattcctatgttggtgggcaccaatattccaagggcagctctctttgctgaatgagggccttctcgtgaggtactgacaacaccagcaactcgagagctgggaaactcaaggaggaggaagaaaatcaaaaccaccctactccctggggtgaggaaagaacaggaggggaggaggaggagtgtgctctgcacttgccttgctccagcatggggtggcagcagaggaatgtcacaggtcagcagcctaggcctccagctataaatagtccggagggcccgagaggctcccgcccgtccagcagggctgtcagcttcctgtgtggcagcacctggcacactggctctggccagcattatgctaaaaccctgttactctccaatgaaccagggaggcgggctcctctctgcgcctatcccttgagaattctgtcttaccagtgaagggtggggctgcccagatcaggcagcaggagtgaggggcacagtcaccccaggccttgctgagcccaggtctgggtactgagtgtccagagctgcctccccaggaggttaaggtgggggcaaaggggaagcttcaagcactttgcctacttttgttcactccccagtgcactgtgactcaggccttcccatcaggcctatttgtctacccaataaagcgtgttttttccagaaaaaaaaaaaaaaaaaaaaaaaaaaaaaaaaaaa

SEQ ID NO: 8 - protein, 349 aa, Homo sapiens
MASKKVCIVGSGNWGSAIAKIVGGNAAQLAQFDPRVTMWVFEEDIGGKKLTEIINTQHENVKYLPGHKLPPNVVAVPDVVQAAEDADILIFVVPHQFIGKICDQLKGHLKANATGISLIKGVDEGPNGLKLISEVIGERLGIPMSVLMGANIASEVADEKFCETTIGCKDPAQGQLLKELMQTPNFRITVVQEVDTVEICGALKNVVAVGAGFCDGLGFGDNTKAAVIRLGLMEMIAFAKLFCSGPVSSATFLESCGVADLITTCYGGRNRKVAEAFARTGKSIEQLEKELLNGQKLQGPETARELYSILQHKGLVDKFPLFMAVYKVCYEGQPVGEFIHCLQNHPEHM